(19) United States
(12) Patent Application Publication
(10) Pub. No.: US 2015/0216926 A1
(43) Pub. Date: Aug. 6, 2015

(54) METHODS OF REACTIVATING A LATENT HUMAN IMMUNODEFICIENCY VIRUS

(71) Applicant: THE UAB RESEARCH FOUNDATION, Birmingham, AL (US)

(72) Inventor: Olaf Kutsch, Birmingham, AL (US)

(21) Appl. No.: 14/316,144

(22) Filed: Jun. 26, 2014

Related U.S. Application Data
(60) Provisional application No. 61/839,499, filed on Jun. 26, 2013.

(51) Int. Cl. A61K 38/07; A61K 31/145; A61K 38/15; A61K 31/22; A61K 45/06; A61K 38/05; A61K 31/365

(52) U.S. Cl. CPC A61K 38/07; A61K 38/05; A61K 31/145; A61K 31/365; A61K 31/22; A61K 45/06; A61K 38/15

(57) ABSTRACT

Provided herein are methods of reactivating a latent Human Immunodeficiency Virus (HIV) infection in a cell or subject. The methods include contacting the cell with or administering to the subject one or more proteasome inhibitors, and contacting the cell with or administering to the subject one or more reactivating agents. Also provided are methods of treating an HIV infection in a subject. The methods include administering to the subject one or more proteasome inhibitors, administering to the subject one or more reactivating agents and administering to the subject one or more anti-retroviral agents.

This application claims the benefit of U.S. Provisional Application No. 61/839,499, filed Jun. 26, 2013, which is hereby incorporated herein in its entirety.

### STATEMENT REGARDING FEDERALLY FUNDED RESEARCH

This invention was made with government support under Grant No. RO1AI077457 awarded by the National Institutes of Health (NIH). The government has certain rights in the invention.

### BACKGROUND

Eradication of the latent HIV-1 reservoir is considered a key requirement to the cure of an HIV-1 infection. However, the molecular mechanisms controlling latent HIV-1 infection are incompletely understood, making it difficult to develop efficient and targeted therapeutics. It is widely assumed that HIV-1 latency is the result of a restrictive chromatin environment on the viral promoter. This idea has guided the majority of the therapeutic efforts to eradicate the latent HIV-1 reservoir in which histone deacetylase (HDAC) inhibitors were used to relieve this transcriptional restriction. Some of these trials showed limited promise, as the drugs were found to induce transient viremia. However, HDAC inhibitors in patient-derived ex vivo material were shown to have limited or no HIV-1 reactivating effect.

### SUMMARY

Provided herein are methods of reactivating a latent Human Immunodeficiency Virus (HIV) infection in a cell or subject. The methods include contacting the cell with or administering to the subject one or more proteasome inhibitors and one or more reactivating agents.

Also provided are methods of treating an HIV infection in a subject. The methods include administering to the subject one or more proteasome inhibitors, one or more reactivating agents and one or more anti-retroviral agents.

The details of one or more embodiments are set forth in the accompanying drawings and the description below. Other features, objects, and advantages of the invention will be apparent from the description and drawings, and from the claims.

### DESCRIPTION OF DRAWINGS

FIGS. 1A, 1B and 1C show phenotypic changes in latently HIV-1 infected host T cells, suggesting a potential functional correlation of latent HIV-1 infection with T cell anergy. FIG. 1A shows the results of flow cytometric analysis indicating, relative to the parental Jurkat T cells, alterations in cell surface expression of several relevant proteins on latently infected T cells. While expression of CD3 or CD28 was down-regulated, expression of some tetraspanins (here CD9) was upregulated. FIG. 1B is a graph showing kinetic NF-κB p50 activation profile following PMA stimulation as determined by TransAM assays for Jurkat (not infected) and latently HIV-1 infected CA5 T cells. FIG. 1C is a graph showing kinetic NF-κB p65 activation profile following PMA stimulation as determined by TransAM assays for Jurkat (not infected) and latently HIV-1 infected CA5 T cells.

FIGS. 2A, 2B, and 2C show targeting anergy-related mechanisms alters HIV-1 latency control. FIG. 2A is a graph showing pretreatment of J2574 reporter T cells with the T cell anergy inducing Ca²⁺ ionophore ionomycin (black) promotes the establishment of latent HIV-1 infection relative to untreated control cells (gray). Increased protein-ubiquitination for targeting to the proteasome by the E3 ubiquitin ligase GRAIL is a hallmark of T cell anergy. FIGS. 2B and 2C are graphs showing that twenty-four (24) hour pretreatment of the latently HIV-1 infected CA5 T cells with the FDA-approved proteasome inhibitor bortezomib (5 nM) mobilizes latent HIV-1 infection and reduces the activator concentrations that are required to trigger full HIV-1 reactivation at the population level. FIG. 2B is a graph showing bortezomib in combination with the activator bryostatin. FIG. 2C is a graph showing bortezomib in combination with the activator prostratin.

FIG. 3 is a graph showing the proteasome inhibitor bortezomib and disulfiram (AP-1 activator) synergistically trigger HIV-1 reactivation. CA5 T cells were pretreated for 24 hours with bortezomib. Disulfiram was titrated on control cells or bortezomib pretreated T cells. Reactivation was measured 24 hours after disulfiram addition by determining the percentage of GFP-positive cells using flow cytometric analysis.

FIG. 4 is a graph showing the proteasome inhibitor carfilzomib and bryostatin trigger HIV-1 reactivation. CA5 T cells were pretreated for 24 hours with carfilzomib. Bryostatin was titrated on control cells or carfilzomib pretreated T cells. Reactivation was measured 24 hours after bryostatin addition by determining the percentage of GFP-positive cells using flow cytometric analysis.

### DETAILED DESCRIPTION

Anti-retroviral therapy (ART) can suppress, but not eradicate, an HIV-1 infection, as the virus can integrate itself in a dormant or latent state into the genome of long-lived immune cells. The integrated virus persists indefinitely and propagates if therapy is halted. It is believed that the most promising way to eradicate latent HIV-1 infection is to reactivate these latent viruses. Infected cells with reactivated virus could become susceptible to destruction by the immune system, could be destroyed by viral cytotoxicity, or could be therapeutically targeted by HIV-specific agents, thereby deleting this source of residual virus. Unfortunately, some stimuli considered to reactivate latent HIV-1 infection can potentially cause hypercytokinemia, a fatal “cytokine storm.” The methods provided herein, however, reactivate a latent Human Immunodeficiency Virus (HIV) without producing a cytokine storm.

Previous drug screens for HIV-1 reactivating compounds or previous attempts to therapeutically reactivate latent HIV-1 infection in patients were developed under the “one-drug one-target” hypothesis, which is based on the premise that the perfect chemical probe acts on a single target. However, multiple components should be triggered in coordinated fashion to induce HIV-1 reactivation in the absence of sustained T cell activation. This takes into consideration that all genes function in the context of other genes or that molecular control mechanisms function in the context of a network and that there really cannot be a single target, as biological systems respond dynamically and variably based on the activities of interacting genes or mechanisms. Thus, the methods provided herein optionally use combinations of drugs to reactivate latent HIV infections.

Provided herein are methods of reactivating a latent Human Immunodeficiency Virus (HIV) infection in a cell. The methods include contacting the cell with one or more proteasome inhibitors, and contacting the cell with one or more reactivating agents, wherein the reactivating agent reactivates the latent HIV infection in the cell. Contact can occur in vitro or in vivo. Also provided are methods of reactivating a latent Human Immunodeficiency Virus (HIV) infection in a subject. The methods include administering to the subject one or more proteasome inhibitors, and administering to the subject one or more reactivating agents, wherein the reactivating agent reactivates the latent HIV infection in the subject.

Further provided are methods of treating an HIV infection in a subject. The methods include administering to the subject one or more proteasome inhibitors, administering to the subject one or more reactivating agents, and administering to the subject one or more anti-retroviral agents, wherein administration of the anti-retroviral agent treats the HIV infection. Optionally, the anti-retroviral agent is administered to the subject before, during and/or after reactivation of the latent HIV infection. Anti-retroviral agents for use in the provided methods include, but are not limited to, a nucleoside, a nucleoside reverse transcriptase inhibitor (NRTI), a non-nucleoside reverse transcriptase inhibitor (NNRTI), a nucleoside analog reverse transcriptase inhibitor (NARTI), a protease inhibitor, an integrase inhibitor, an entry inhibitor, a maturation inhibitor, and combinations thereof. If the anti-retroviral agent is administered prior to reactivation of the latent HIV infection, the reactivation should occur within the therapeutic window of the anti-retroviral agent and/or the anti-retroviral agent should be administered both before and after reactivation.

In the methods provided herein, the use of the provided proteasome inhibitors reduce the amount or number of doses of the reactivating agent required to reactivate the latent HIV infection in the cell or the subject. The provided proteasome inhibitors serve to prime the latent HIV infection for reactivation, e.g., by lowering the activation threshold for latent infection. Full reactivation can then be triggered by a reactivating factor, which by itself at a low dose would have little or no effect on latent infection, and most importantly, would not trigger or would trigger only minimal cytokine expression. For example, priming the latent HIV infection with the provided proteasome inhibitors or second priming agents can affect modulation of NF-κB activity by the reactivating agent that avoids triggering a “cytokine storm.” Thus, administration of the proteasome inhibitor reduces the amount (i.e., dosage) of the agent needed to reactivate the latent HIV infection (i.e., reactivation agent) in the cell or subject. By way of example, the dosage amount of reactivating agent can be in the range of one log (factor 10) less that the amount of the agent required in the absence of the proteasome inhibitor. However, the effective dosage of reactivating agent may be reduced by a factor of 1, 2, 3, 4, 5, 6, 7, 8, 9, 10 times or more in the presence of the proteasome inhibitor.

The term latent, as used herein, in the context of a latent immunodeficiency virus refers to a genomically integrated immunodeficiency virus (including a latent immunodeficiency virus-based retroviral vector, e.g., a recombinant immunodeficiency virus) that is transcriptionally silent or inactive, for example immunodeficiency virus transcripts are undetectable or are at background levels in a cell comprising the latent immunodeficiency virus.

The terms reactivated and reactivation, as used herein, refers to an immunodeficiency virus that, after a period of latency, becomes transcriptionally active. By way of example, the virus is transcriptionally active if it has a functional Tat protein mediates transcription from a functional immunodeficiency virus promoter (e.g., a long terminal repeat promoter). The reactivated virus can form infectious viral particles. As used herein, activation and activated mean the same as reactivation and reactivated, respectively and the terms are used interchangeably herein. For example, a latent immunodeficiency virus that can be activated also means a latent immunodeficiency virus that can be reactivated as these terms both convey the state of transcriptional activity of an immunodeficiency virus, as opposed to a state of latency characterized by transcriptional inactivity.

The term immunodeficiency virus, as used herein, refers to human immunodeficiency virus-1 (HIV-1); human immunodeficiency virus-2 (HIV-2); and any of a variety of HIV subtypes and quasispecies. The methods and compositions disclosed herein are also applicable to simian immunodeficiency virus (SIV) and feline immunodeficiency virus (FIV). Thus, although HIV is used by way of example throughout, SIV and FIV could be treated with the same methods and compositions as HIV. Optionally, the latent replication competent or non-replication competent immunodeficiency virus can be human immunodeficiency virus (HIV). For example, the immunodeficiency virus can be HIV-1 or HIV-2.

The proteasome is a protein complex involved in the degradation of proteins that are damaged or unneeded or unwanted. The proteasome and its subunits are referred to by sedimentation coefficient (denoted S). The proteasome most often found in mammals is the 26S proteasome made up of a 20S protein subunit and two 19S subunits. Optionally, the proteasome inhibitor is an inhibitor of the 26S proteasome. Optionally, the proteasome inhibitor is an inhibitor of the 20S subunit or the 19S subunit. Optionally, the proteasome inhibitor is not an inhibitor of the 20S subunit. Proteasome inhibitors for use in the provided methods include, but are not limited to, bortezomib, carfilzomib, epigallocatechin-3-gallate, salinosporamide A, ONX-0912, CEP-18770, MLN9708, epoxomicin, and MG132. See, e.g., Osanai et al., Bioorg. Med. Chem. 15(15):5076-82 (2007); Meng et al., PNAS 96:10403-8 (1999); and Lonial et al., Oncology, Supplement Volume 25 No. 2 (2011); Gallerani et al., Eur. J. Cancer 49(2):290-6 (2013); and Micel et al., J. Clin. Oncol. 31(9):1231-8 (2013), which are incorporated by reference herein in their entireties. Optionally, the proteasome inhibitor is not aclacinomycin. Optionally, the proteasome inhibitor is bortezomib or carfilzomib.

Reactivating agents for use in the provided methods include, but are not limited to, antibodies, peptides, and chemical compounds. Optionally, the reactivating agents include, but are not limited to, protein kinase C (PKC) activators, NF-κB or NF-κB pathway activators, bacterial peptides, and glycolipids. Optionally, the reactivating agent is TNF-α, IL-2 or a CD3 antibody. Optionally, the reactivating agent is selected from the group consisting of disulfiram, bryostratin, and prostratin.

Optionally, the reactivating agent is a protein kinase C (PKC) activator or an NF-κB pathway activator. Optionally, the NF-κB activator modulates the level of NF-κB activity. Optionally, the NF-κB activator results in a transient first increase in the level of NF-κB activity without a delayed second increase in NF-κB activity. Thus, the transient first increase in the level of NF-κB activity is not followed by a sustained level of NF-κB activity. A sustained level of NF-κB activity, can, for example, result in the induction of cytokine gene expression and a concomitant delayed increase. As described herein, the NF-κB activator produces a transient first increase in the level of NF-κB activity, resulting in a peak level of NF-κB activity, with the level of NF-κB subsequently decreasing over time. Little or no second peak of activity occurs. The delayed second increase in NF-κB activity may be associated with cytokine gene induction. The absence or reduction of a delayed second increase in NF-κB activity results in the absence of substantial cytokine gene induction. Optionally, the absence of cytokine gene induction comprises the absence of substantial induction of one or more of TNF-α, IL-8, IFNγ, IL-2, IL-4, and IL-6. By substantial cytokine gene induction is meant an increase over control that is significantly higher than control values using standard statistical analysis. The modulation of NF-κB activity differs in pattern from a modulation caused by TNF-α, PMA, PHA-L, IL-2, anti-CD3 monoclonal antibodies, or a combination of anti-CD-3 and anti-CD28 monoclonal antibodies. The modulation of NF-κB activity caused by TNF-α, PMA, PHA-L, IL-2, anti-CD3 monoclonal antibodies, or a combination of anti-CD-3 and anti-CD28 monoclonal antibodies can, for example, produce a pattern of NF-κB activity. Optionally, the pattern of NF-κB activity caused by these agents begins with a first increase in the level of NF-κB activity, followed by a sustained increased level of NF-κB activity. The sustained level of NF-κB activity can, for example, be an oscillating level of NF-κB activity. An oscillating pattern of NF-κB activity includes an increase in level of NF-κB activity, a decrease in level of NF-κB activity, and another increase, but the pattern can continue to repeat.

Optionally, the reactivating agent is a flagellin polypeptide or fragment thereof. Optionally, the flagellin polypeptide or fragment thereof comprises a bacterial flagellin polypeptide or fragment thereof. Optionally, the bacterial flagellin polypeptide or fragment thereof is a Massilia flagellin polypeptide or fragment thereof, for example, a Massilia timonae flagellin polypeptide or fragment thereof. Optionally, the bacterial flagellin polypeptide or fragment thereof is selected from a Salmonella flagellin polypeptide, an E. coli flagellin polypeptide, or fragment thereof. Optionally, the Salmonella flagellin polypeptide or fragment thereof is a Salmonella typhimurium flagellin polypeptide or fragment thereof. Optionally, the E. coli flagellin polypeptide or fragment thereof is an E. coli K12 flagellin polypeptide or fragment thereof. Optionally, the bacterial peptide comprises SEQ ID No:2, 3, or 4 or a fragment of SEQ ID No:2, 3, or 4. Reactivating agents and methods of using reactivating agents are described in International Publication Nos. WO 2013/074794 and WO 2011/146612, which are incorporated by reference herein in their entireties.

Methods for detecting latent virus reactivation are described in, for example, International Publication No. WO 2006/029029, which is incorporated by reference herein in its entirety.

Optionally, the latent HIV infection is primed in the cell by administration of a second agent or second priming agent. The second agent can, for example, comprise an anthracycline or a nucleoside analogue. Optionally, the second agent is selected from the group consisting of actinomycin D, ampotericin B, WP631, a retinoid, dactinomycin, cytarabine, and 5′-azacytidine. Optionally, the second agent is a cell differentiation activator or a cell reprogramming factor.

In any of the methods set forth herein, the retinoid can be, for example, a compound of Formula I

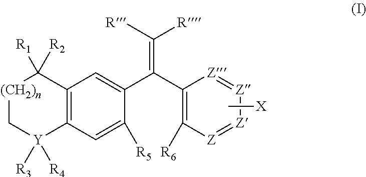

wherein

R₁ and R₂ each independently represent hydrogen or lower alkyl or acyl having 1-4 carbon atoms;

Y represents C, O, S, N, CHOH, CO, SO, SO₂ or a pharmaceutically acceptable salt;

R₃ represents hydrogen or lower alkyl having 1-4 carbon atoms where Y is C or N;

R₄ represents hydrogen or lower alkyl having 1-4 carbon atoms where Y is C, but R₄ does not exist if Y is N, and neither R₃ or R₄ exist if Y is S, O, CHOH, CO, SO, or SO₂;

R′″ and R″″ represent hydrogen, halogen, lower alkyl or acyl having 1-4 carbon atoms, alkyl amino, or R′″ and R″″ taken together form a cycloalkyl group having 3-10 carbons, and wherein the cycloalkyl group can be substituted with lower alkyl having 1-4 carbons or halogen;

R₅ represents hydrogen, a lower alkyl having 1-4 carbons, halogen, nitro, OR₇, SR₇, NR₇R₈ or (CF)ₙCF₃;

Z, Z′, Z″ and Z′″ are all carbon; and

X is COOH, tetrazole, PO₃H, SO₃H, CHO, CH₂OH, CONH₂, COSH, COOR₉, COSR₉, CONHR9, or COOW, where W is a pharmaceutically acceptable salt, and where X can originate from any C or N on the ring. For example, a compound of Formula I can be a compound of Formula II

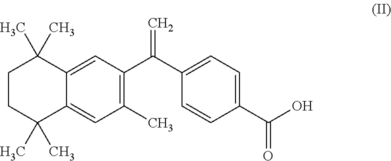 [SMILES: C=C(c1ccc(C(=O)O)cc1)c1cc2c(cc1C)C(C)(C)CCC2(C)C]

The compound of Formula II is also known as bexarotene or Targretin. Methods of making the compounds of Formula I or Formula II are set forth in U.S. Pat. No. 5,780,676, which is incorporated herein in its entirety by this reference.

The retinoid can also be a compound of Formula IIIa;

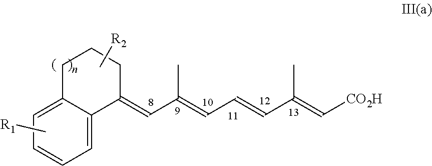

a compound of Formula IIIb;

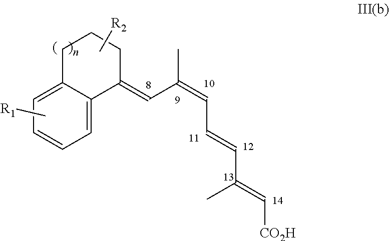

a compound of Formula IVa; or

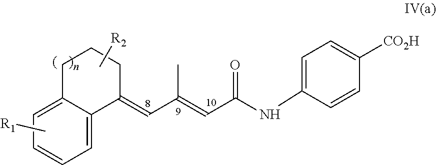

a compound of Formula IVb;

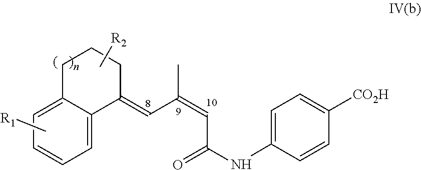

wherein R₁ represents one or two substituents on the aryl ring and is selected from the group consisting of H, ethyl, methyl, n-propyl, i-propyl, t-butyl, phenyl, benzyl, chloro, fluoro, methoxy, ethoxy, benzyloxy, C1-C8 cyclic alkyls, aryl, arylalkyl, alkyloxy, aryloxy, arylalkyloxy, and halogen;

R₂ is selected from the group consisting of H, ethyl, methyl, n-propyl, i-propyl, 2-methylpropyl, n-butyl, cyclohexyl, 3-cyclohexenyl, benzyl, methoxy, ethoxy, benzyloxy, C1-C8 cyclic alkyls, aryl, arylalkyl, alkyloxy, aryloxy and arylalkyloxy; and n=0-3.

For example, a compound of Formula III(a) is a compound of Formula V

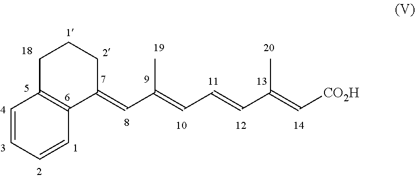 [SMILES: CC(=C\C=C\C(C)=C\C(=O)O)/C=C1\CCCc2ccccc21]

The compound of Formula V is also known as all-trans UAB30.

In another example, a compound of Formula III(b) is a compound of Formula VI

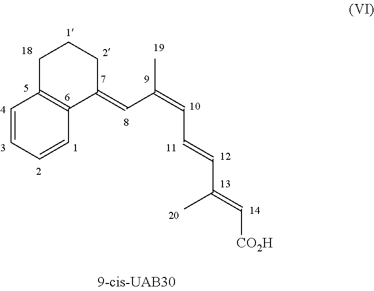 [SMILES: CC(=C/C=C/C(C)=C/C(=O)O)/C=C1\CCCc2ccccc21]

The compound of Formula VI is also known as 9-cis-UAB30.

The cell differentiation activator can, for example, be selected from the group consisting of deferoxamine, haringtonine, mytomycin, bleomycin, methotrexate, purine and pyrimidine analogs, 6-thioguanine, tunicamycin, marcellomycin, or musettamycin.

Provided herein are compositions containing one or more proteasome inhibitors, one or more reactivating agents, one or more second priming agents, one or more anti-retroviral agents, and combinations thereof and a pharmaceutically acceptable carrier. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the proteasome inhibitor, second priming agent, reactivating agent and/or anti-retroviral agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy.

Preparations for parenteral administration include sterile aqueous or non-aqueous solutions, suspensions, and emulsions. Examples of non-aqueous solvents are propylene glycol, polyethylene glycol, oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Compositions and formulations for oral administration include, but are not limited to, powders or granules, microparticulates, nanoparticulates, suspensions or solutions in water or non-aqueous media, capsules, gel capsules, sachets, and tablets. Such compositions may also include thickeners, flavoring agents, diluents, emulsifiers, dispersing aids or binders.

Any of the agents provided herein (e.g., proteasome inhibitor, second priming agent, reactivating agent or anti-retroviral agent) can be used in any combination. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

The provided compositions can be administered one or more times daily, weekly or monthly. Optionally, in the provided methods, the proteasome inhibitor is provided, e.g., contacted with a cell or administered to a subject, prior to the reactivating agent. Optionally, the proteasome inhibitor is provided one or more times prior to the reactivating agent. Optionally, the proteasome inhibitor is provided one or more times for one or more weeks prior to the reactivating agent. Optionally, the proteasome inhibitor is provided one or more times daily for one or more weeks prior to the reactivating agent. Optionally, the proteasome inhibitor is provided one or more times starting two weeks prior to the reactivating agent. Optionally, the proteasome inhibitor is provided one or more times within 96, 72, 48, 24, or 12 hours prior to the reactivating agent.

The provided methods and agents as described herein are useful for therapeutic treatment. Therapeutic treatment involves administering to a subject a therapeutically effective amount of the agents described herein after diagnosis of HIV infection. The terms effective amount and effective dosage are used interchangeably. The term effective amount is defined as any amount necessary to produce a desired physiologic response (e.g., an effective amount of a reactivating agent reactivates a latent HIV infection in at least about 50% of the total cell population; an effective amount of a priming agent primes a latent HIV infection by reducing the effective amount of the reactivating agent needed to reactive a latent HIV infection; and an effective amount of an anti-retroviral agent results in a reduction in HIV viral load 30-100 fold within six weeks with the viral load falling below detectable limits within 4-6 months). Effective amounts and schedules for administering the agent may be determined empirically, and making such determinations is within the skill in the art. The dosage ranges for administration are those large enough to produce the desired effect (e.g., HIV reactivation and/or reduction of HIV symptoms). The dosage should not be so large as to cause substantial adverse side effects, such as unwanted cross-reactions, anaphylactic reactions, and the like. Dosages of reactivating agent can, for example, be reduced by using a proteasome inhibitor. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products.

Provided herein are kits comprising one or more of the provided compositions and instructions for use. Optionally, the kit comprises one or more doses of an effective amount of a composition comprising a one or more proteasome inhibitors, one or more reactivating agents, one or more second priming agents, one or more anti-retroviral agents or combinations thereof. Optionally, the kit comprises one or more doses of a proteasome inhibitor and one or more doses of a reactivating agent. Optionally, the proteasome inhibitor and reactivating agent are in different containers. The kits can further include one or more doses of an anti-retroviral agent. Optionally, the compositions in the kit are present in a container (e.g., vial or packet). Optionally, the kit comprises a means of administering the compositions, such as, for example, a syringe, needle, tubing, catheter, patch, and the like. The kit may also comprise formulations and/or materials requiring sterilization and/or dilution prior to use.

As used herein the terms treatment, treat, or treating refers to a method of reducing or delaying the effects of a disease or condition (e.g., HIV infection) or symptom of the disease or condition (e.g., treatment results in an increase in CD4⁺ T cells and a reduction in HIV viral load). Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLE

### Example 1

### Proteasome Inhibitor and Reactivating Agent Combinations for Triggering HIV Reactivation

Consistent with the idea that kinase activity plays a key role in the control of latent infection, kinase activity profiling of several latently HIV-1 infected T cell lines revealed that latently infected T cells exhibit a strikingly different kinase activity profile relative to the parental Jurkat cells. The kinase activity profile for the latently HIV-1 infected EF7 and CA5 T cells was determined, as these cell lines are well defined regarding viral integration in actively expressed host genes and >90% reactivation can be achieved at the population level using PMA stimulation (Duverger et al., Journal of Virology 83:3078-93 (2009); Kutsch et al., Journal of Virology 78:8776-8786 (2002); Shishido et al., Journal of Virology 89:9055-9069 (2012); and Wolschendorf et al., Journal of Virology 84:8712-20 (2010)).

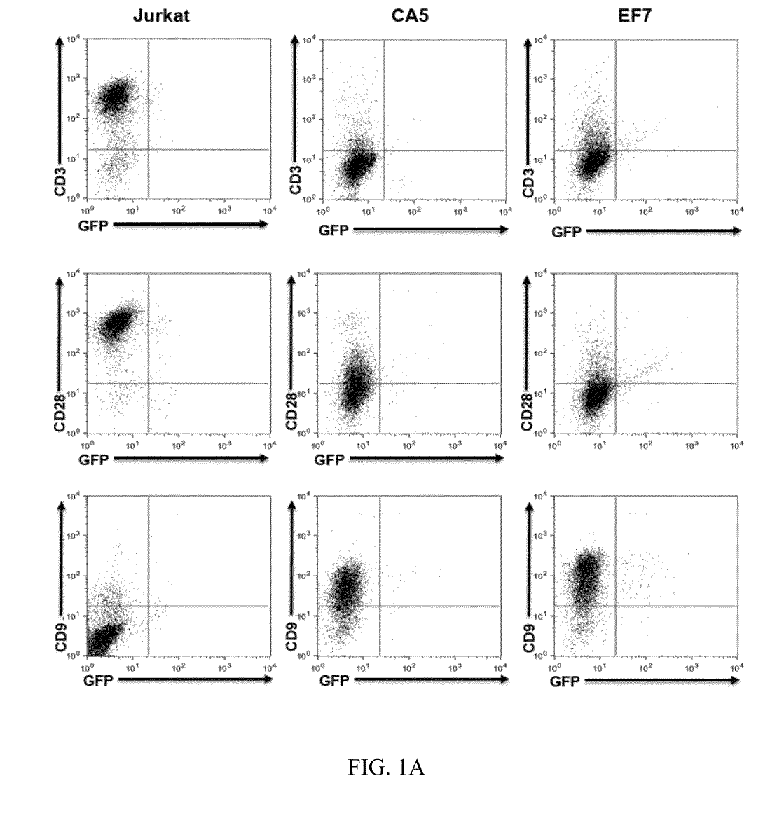

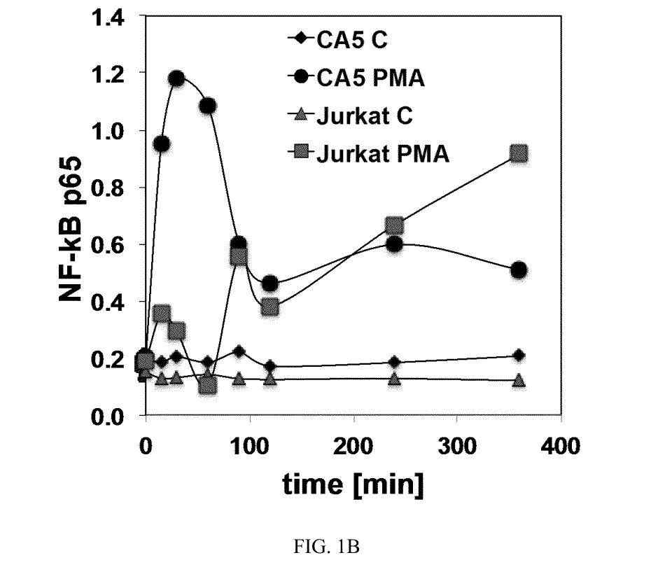

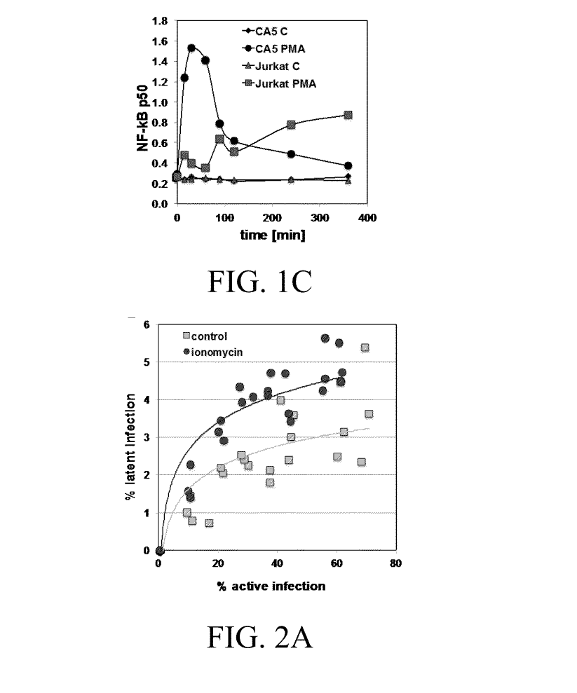

The observed differences in the kinase activity profiles of latently infected T cells relative to the parental cells were correlated with differences in the cell surface protein expression profile (FIG. 1A). Most notably, CD3 and CD28 were down-regulated on the tested latently infected T cell lines, whereas some tetraspanins, e.g. CD9 and CD151, were found to be expressed at higher levels. Other cell surface molecules, such as CD4, were not regulated differentially.

The observed phenotypic changes in latently infected T cells were further reflected in the kinetic NF-κB activation response to stimulation with phorbol 12-myristate 13-acetate (PMA). Whereas PMA stimulation induced a classic sinus-wave shaped kinetic NF-κB activation response profile with an increasing amplitude, the kinetic NF-κB response in all latently infected T cell clones (total of 5 clones; FIG. 1B) was characterized by a rapid and massively overshooting initial amplitude and a reduction of the subsequent, sustained increase in NF-κB activity.

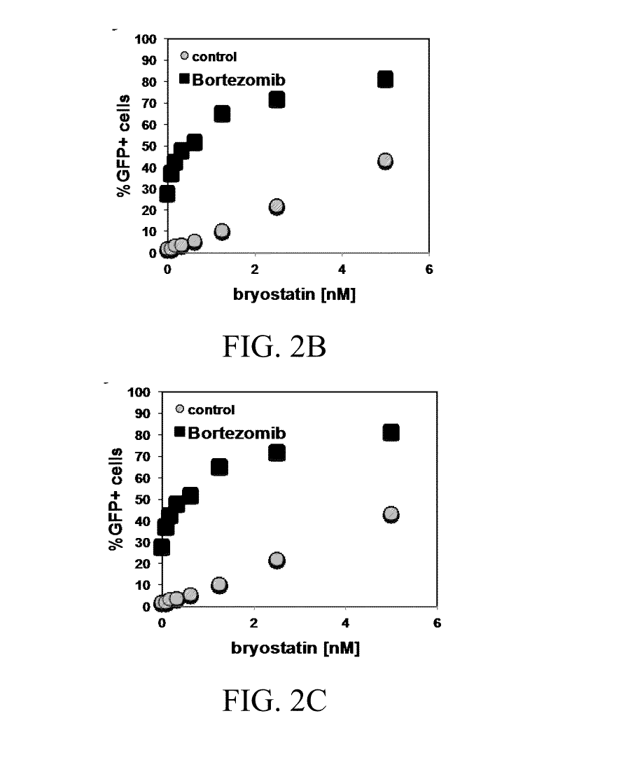

Without being bound by any theory, a working concept derived from the altered cellular phenotype observed in latently HIV-1 infected T cells could be that HIV-1 latency is correlated with the induction of a pseudoanergic state of the host-cell. HIV-1 infection has been reported to be associated with high levels of induced T cell anergy. Exposure to gp120 has been reported to be sufficient to trigger T cell anergy (Bouhdoud et al., Journal of Virology 74:2121-2130 (2000); Dybul et al., J. Immunol. 165:1685-91 (2000); Masci et al., Journal of Leukocyte Biology 74:1117-1124 (2003); and Schols and De Clercq, Journal of Virology 70:4953-4960 (1996)). Also, other retroviruses are reported to induce epigenetic changes in their host cells. Ubiquitin and CDK2 are also known to be involved in T cell anergy (Greenwald et al., Immunity 14:145-155 (2001); and Li et al., Nature Immunology 7:1157-65 (2006)). Both factors are directly connected to kinases (PIM-1 and JNK) that are demonstrated to be important for HIV-1 latency control. It was thus tested whether the Ca²⁺ ionophore ionomycin, an inducer of T cell anergy in primary T cells (Macian et al., Cell 109:719-31 (2002)) and in Jurkat T cells (Telander et al., J. Immunol. 162:1460-5 (1999)), would affect the ability of HIV-1 to establish latent HIV-1. Pretreatment of J2574 T cells with ionomycin almost doubled the amount of established stable latent infection events, independent of the level of virus input (FIG. 2A).

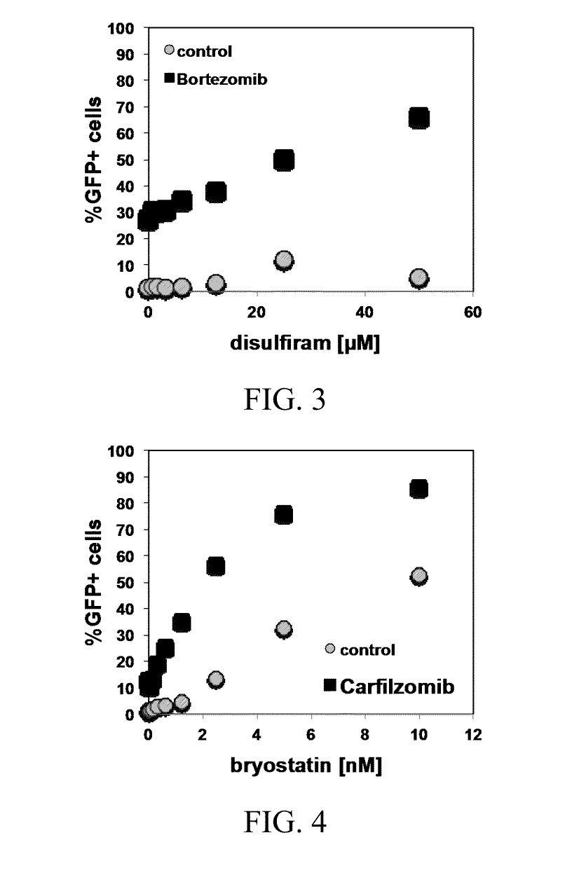

As mentioned above, T cell anergy has been reported to be associated with a series of changes in the kinase activity profile. A hallmark of T cell anergy is the expression of GRAIL (gene related to anergy in lymphocytes), a trans-membrane RING finger ubiquitin E3 ligase (Anandasabapathy et al., Immunity 18:535-547 (2003)). As other ubiquitin ligases, GRAIL tags proteins for degradation by the proteasome. Without being bound by any theory, it was reasoned that, if increased protein ubiquitination by ubiquitin ligases contribute to the control of latent HIV-1 infection, then proteasome inhibitors may be useful to mobilize latent HIV-1 infection. In order to determine this, two FDA-approved proteasome inhibitors, bortezomib and carfilzomib, were tested for their ability to mobilize latent HIV-1 infection, either by themselves, or in combination with potentially therapeutically relevant known HIV-1 reactivating activators, such as bryostatin, prostratin, and disulfiram. As seen in FIG. 2B, bortezomib (5 nM) by itself triggered mobilization of latent HIV-1 infection in ˜30% of the latently infected T cells and acted highly synergistic with bryostatin. Bortezomib also interacted with prostratin (FIG. 2C) and disulfiram (FIG. 3) to trigger HIV-1 reactivation. Carfilzomib by itself triggered HIV-1 reactivation and also potently interacted with bryostatin (FIG. 4).

These data indicated that proteasome inhibitors mobilize latent HIV infection and reduce activator concentrations required to trigger full HIV reactivation.

A number of embodiments have been described. Nevertheless, it will be understood that various modifications may be made. Accordingly, other embodiments are within the scope of the following claims.

SEQ ID NO: 1
Exemplary Bacterial Reactivating Factor DNA sequence
atggcatccgtaatcaataccaacgttccttccctgaattcgcagcgtaacctgtcgacctcgcaagcacagctgaac
acctcgattcagcgtctgtcgtcgggcatgcgcatcaacagcgcgaaggacgacgccgccggcctcgcgatttccgatcgcat
gaattcgcagatcaagggcatgacccaggctacccgtaacgccaacgatggcgtgtcgatggcccagaccgccgaaggcgct
ctgtcgagctcgggcgacatcctgcagcgtatccgcgaactggcagtgcagtcgtcgaactcgtcgaactcggccagcgaccg
caaggccctgcagaccgaagttacccaactgagctcggaactgaatcgcatcgccaacaccaccgagttcaacggccagaag
ctgatggacggcaccctgggtaccgcgaatttccaggtcggcgccaatgcggggcagctgatctcgatgaccggcgcgaactt
caacacctcggtgtacggcaacaaccagatcgctggcgatgccgcgaaggcagcagccacgacgtcggcggccgccactg
gagcatttaccatttcaggctacctcggcacgagcgccgcgattaacacgaccgatgctgacacggcaaagaccatcgccgcg
agcatcaacaacgtcaccggcgatactggtgtcaccgcaaccgcccgtaccgattcgttcctcaagagcgcaggcggtgcgca
taccatgagcctggcctcggacaactcgggtaccggcgaagctgtcgacatctcgttcactgtcggtgacggcggcgatacgg
ccgacgactacgcggcagccatcagcgccttcaacgcacaaaccgcgaagactggcgtgaccgccgagtatgatgcagtcg
aaaaaggtctcaagctgaccaactcggccggtgaaaacatttcgctgaccaacaaagccggtagcgccgatatcgacatggac
acctacgccgccgacggcgcgctgatggcctccgcactcaccctggaggctggcgacgttggcgtggccaacggtcgcgtg
accatggactccgagaagagcttctcggtggccgaaggcaccgatacgggttttgaactcgcggattcgtcagccttgaaatcg
gtcgccaagatcgacatcagcgacttcgagggcgcacaggaagccatcaaggtcgccgatgccgccctctcggccgtcaaca
gccagcgcgctgaatacggcgcactgcagtcgcgcttcgaatcggcgatctcgaacctgtcgtcgtcgaccgagaacctgtcg
gcatcgcgcagccgcatcgtcgataccgacttcgcagctgaaaccgcaaagatgacccgcggccagatcctgcagcaagctg
gtacctcgatgctggctcaggctaactccctgccgaacggtgtcctgtcgctgctgcgtggc
SEQ ID NO: 2
Exemplary Bacterial Reactivating Factor polypeptide sequence
MASVINTNVPSLNSQRNLSTSQAQLNTSIQRLSSGMRINSAKDDAAGLAIS
DRMNSQIKGMTQATRNANDGVSMAQTAEGALSSSGDILQRIRELAVQSSNSSNSA
SDRKALQTEVTQLSSELNRIANTTEFNGQKLMDGTLGTANFQVGANAGQLISMT
GANFNTSVYGNNQIAGDAAKAAATTSAAATGAFTISGYLGTSAAINTTDADTAK
TIAASINNVTGDTGVTATARTDSFLKSAGGAHTMSLASDNSGTGEAVDISFTVGD
GGDTADDYAAAISAFNAQTAKTGVTAEYDAVEKGLKLTNSAGENISLTNKAGSA
DIDMDTYAADGALMASALTLEAGDVGVANGRVTMDSEKSFSVAEGTDTGFELA
DSSALKSVAKIDISDFEGAQEAIKVADAALSAVNSQRAEYGALQSRFESAISNLSSS
TENLSASRSRIVDTDFAAETAKMTRGQILQQAGTSMLAQANSLPNGVLSLLRG
SEQ ID NO: 3
Polypeptide sequence for Salmonella typhimurium flagellin
MAQVINTNSLSLLTQNNLNKSQSALGTAIERLSSGLRINSAKDDAAGQAIA
NRFTANIKGLTQASRNANDGISIAQTTEGALNEINNNLQRVRELAVQSANSTNSQS
DLDSIQAEITQRLNEIDRVSGQTQFNGVKVLAQDNTLTIQVGANDGETIDIDLKQI
NSQTLGLDTLNVQQKYKVSDTAATVTGYADTTIALDNSTFKASATGLGGTDQKI
DGDLKFDDTTGKYYAKVTVTGGTGKDGYYEVSVDKTNGEVTLAGGATSPLTGG
LPATATEDVKNVQVANADLTEAKAALTAAGVTGTASVVKMSYTDNNGKTIDGGL
AVKVGDDYYSATQNKDGSISINTTKYTADDGTSKTALNKLGGADGKTEVVSIGG
KTYAASKAEGHNFKAQPDLAEAAATTTENPLQKIDAALAQVDTLRSDLGAVQNR
FNSAITNLGNTVNNLTSARSRIEDSDYATEVSNMSRAQILQQAGTSVLAQANQVP
QNVLSLLR
SEQ ID NO: 4
Polypeptide sequence for E. coli K12 flagellin
MAQVINTNSLSLITQNNINKNQSALSSSIERLSSGLRINSAKDDAAGQAIAN
RFTSNIKGLTQAARNANDGISVAQTTEGALSEINNNLQRVRELTVQATTGTNSESD
LSSIQDEIKSRLDEIDRVSGQTQFNGVNVLAKNGSMKIQVGANDNQTITIDLKQID
AKTLGLDGFSVKNNDTVTTSAPVTAFGATTTNNIKLTGITLSTEAATDTGGTNPAS
IEGVYTDNGNDYYAKITGGDNDGKYYAVTVANDGTVTMATGATANATVTDANT
TKATTITSGGTPVQIDNTAGSATANLGAVSLVKLQDSKGNDTDTYALKDTNGNLY
AADVNETTGAVSVKTITYTDSSGAASSPTAVKLGGDDGKTEVVDIDGKTYDSAD
LNGGNLQTGLTAGGEALTAVANGKTTDPLKALDDAIASVDKFRSSLGAVQNRLDS
AVTNLNNTTTNLSEAQSRIQDADYATEVSNMSKAQIIQQAGNSVLAKANQVPQQ
VLSLLQG

1. A method of reactivating a latent Human Immunodeficiency Virus (HIV) infection in a cell, the method comprising the steps of:
(a) contacting the cell with one or more proteasome inhibitors, wherein the proteasome inhibitor is not aclacinomycin; and
(b) contacting the cell with one or more reactivating agents, wherein the reactivating agent reactivates the latent HIV infection in the cell.

2. The method of claim 1, wherein the one or more proteasome inhibitors reduce the amount or number of doses of the reactivating agent required to reactivate the latent HIV infection in the cell

3. The method of claim 1, wherein the contacting steps are performed in vitro.

4. The method of claim 1, wherein the contacting steps are performed in vivo.

5. The method of claim 1, wherein the cell is contacted with the proteasome inhibitor prior to the reactivating agent.

6. (canceled)

7. (canceled)

8. (canceled)

9. A method of reactivating a latent Human Immunodeficiency Virus (HIV) infection in a subject, the method comprising the steps of:
(a) administering to the subject one or more proteasome inhibitors, wherein the proteasome inhibitor is not aclacinomycin; and
(b) administering to the subject one or more reactivating agents, wherein the reactivating agent reactivates the latent HIV infection in the subject.

10. The method of claim 9, wherein the one or more proteasome inhibitors reduce the amount or number of doses of the reactivating agent required to reactivate the latent HIV infection in the subject.

11. (canceled)

12. (canceled)

13. (canceled)

14. (canceled)

15. The method of claim 1, wherein the proteasome inhibitor is selected from the group consisting of bortezomib and carfilzomib.

16. The method of claim 1, wherein the reactivating agent is selected from the group consisting of disulfiram, bryostratin, and prostratin.

17.-27. (canceled)

28. The method of claim 1, wherein the method further comprises administering to the subject one or more second agents that prime the latent HIV infection for reactivation.

29. The method of claim 28, wherein the agent that primes the latent HIV infection for reactivation is selected from the group consisting of actinomycin D, amphotericin B, WP631, a retinoid, dactinomycin, cytarabine, and 5′-azacytidine.

30. The method of claim 29, wherein the agent is actinomycin D and the actinomycin D is administered at a dose of 15 micrograms per kilogram per day (μg/kg/day).

31. The method of claim 29, wherein the agent is a retinoid selected from the group consisting of a compound of Formula I 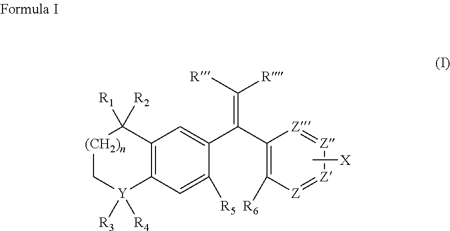
wherein
R₁ and R₂ each independently represent hydrogen or lower alkyl or acyl having 1-4 carbon atoms;
Y represents C, O, S, N, CHOH, CO, SO, SO₂ or a pharmaceutically acceptable salt;
R₃ represents hydrogen or lower alkyl having 1-4 carbon atoms where Y is C or N;
R₄ represents hydrogen or lower alkyl having 1-4 carbon atoms where Y is C, but R₄ does not exist if Y is N, and neither R₃ or R₄ exist if Y is S, O, CHOH, CO, SO, or SO₂;
R′″ and R″″ represent hydrogen, halogen, lower alkyl or acyl having 1-4 carbon atoms, alkyl amino, or R′″ and R″″ taken together form a cycloalkyl group having 3-10 carbons, and wherein the cycloalkyl group can be substituted with lower alkyl having 1-4 carbons or halogen;
R₅ represents hydrogen, a lower alkyl having 1-4 carbons, halogen, nitro, OR₇, SR₇, NR₇R₈ or (CF)ₙCF₃;
Z, Z′, Z″ and Z′″ are all carbon; and
X is COOH, tetrazole, PO₃H, SO₃H, CHO, CH₂OH, CONH₂, COSH, COOR₉, COSR₉, CONHR9, or COOW, where W is a pharmaceutically acceptable salt, and where X can originate from any C or N on the ring;
a compound of Formula III(a); 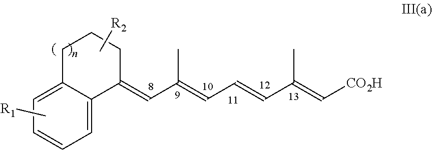
a compound of Formula IIIb; 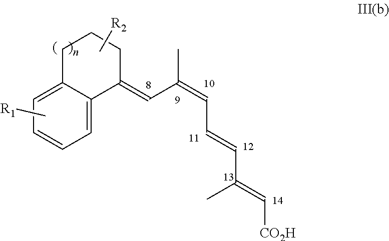
a compound of Formula IVa; and 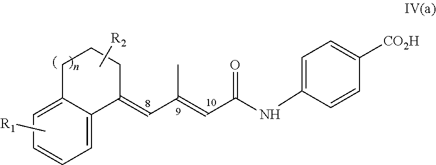
a compound of Formula IVb; 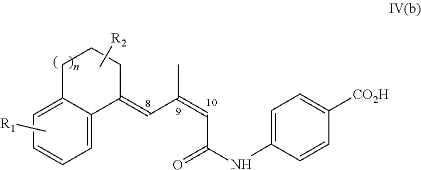
wherein R₁ represents one or two substituents on the aryl ring and is selected from the group consisting of H, ethyl, methyl, n-propyl, i-propyl, t-butyl, phenyl, benzyl, chloro, fluoro, methoxy, ethoxy, benzyloxy, C1-C8 cyclic alkyls, aryl, arylalkyl, alkyloxy, aryloxy, arylalkyloxy, and halogen;
R₂ is selected from the group consisting of H, ethyl, methyl, n-propyl, i-propyl, 2-methylpropyl, n-butyl, cyclohexyl, 3-cyclohexenyl, benzyl, methoxy, ethoxy, benzyloxy, C1-C8 cyclic alkyls, aryl, arylalkyl, alkyloxy, aryloxy and arylalkyloxy; and n=0-3.

32. The method of claim 31, wherein the compound is selected from the group consisting of: a compound of Formula II 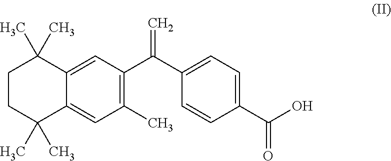 [SMILES: C=C(c1ccc(C(=O)O)cc1)c1cc2c(cc1C)C(C)(C)CCC2(C)C]
a compound of Formula V 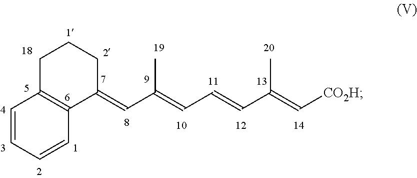 [SMILES: CC(=C\C=C\C(C)=C\C(=O)O)/C=C1\CCCc2ccccc21]
or a compound of Formula VI

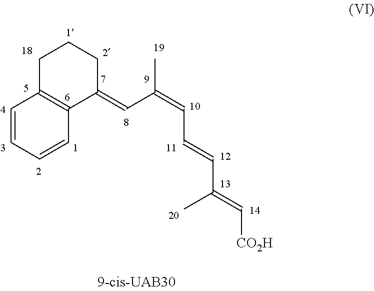 [SMILES: CC(=C/C=C/C(C)=C/C(=O)O)/C=C1\CCCc2ccccc21]

33. The method of claim 28, wherein the agent that primes the latent HIV infection for reactivation is a cell differentiation activator or a cell reprogramming factor.

34. The method of claim 33, wherein the cell differentiation activator is selected from the group consisting of deferoxamine, haringtonine, mytomycin, bleomycin, methotrexate, purine and pyrimidine analogs, 6-thioguanine, tunicamycin, marcellomycin, or musettamycin.

35.-61. (canceled)


SEQUENCE LISTING (4 sequences)

SEQ ID NO: 1 - DNA, 1458 bp, Artificial sequence
  Synthetic construct
atggcatccgtaatcaataccaacgttccttccctgaattcgcagcgtaacctgtcgacctcgcaagcacagctgaacacctcgattcagcgtctgtcgtcgggcatgcgcatcaacagcgcgaaggacgacgccgccggcctcgcgatttccgatcgcatgaattcgcagatcaagggcatgacccaggctacccgtaacgccaacgatggcgtgtcgatggcccagaccgccgaaggcgctctgtcgagctcgggcgacatcctgcagcgtatccgcgaactggcagtgcagtcgtcgaactcgtcgaactcggccagcgaccgcaaggccctgcagaccgaagttacccaactgagctcggaactgaatcgcatcgccaacaccaccgagttcaacggccagaagctgatggacggcaccctgggtaccgcgaatttccaggtcggcgccaatgcggggcagctgatctcgatgaccggcgcgaacttcaacacctcggtgtacggcaacaaccagatcgctggcgatgccgcgaaggcagcagccacgacgtcggcggccgccactggagcatttaccatttcaggctacctcggcacgagcgccgcgattaacacgaccgatgctgacacggcaaagaccatcgccgcgagcatcaacaacgtcaccggcgatactggtgtcaccgcaaccgcccgtaccgattcgttcctcaagagcgcaggcggtgcgcataccatgagcctggcctcggacaactcgggtaccggcgaagctgtcgacatctcgttcactgtcggtgacggcggcgatacggccgacgactacgcggcagccatcagcgccttcaacgcacaaaccgcgaagactggcgtgaccgccgagtatgatgcagtcgaaaaaggtctcaagctgaccaactcggccggtgaaaacatttcgctgaccaacaaagccggtagcgccgatatcgacatggacacctacgccgccgacggcgcgctgatggcctccgcactcaccctggaggctggcgacgttggcgtggccaacggtcgcgtgaccatggactccgagaagagcttctcggtggccgaaggcaccgatacgggttttgaactcgcggattcgtcagccttgaaatcggtcgccaagatcgacatcagcgacttcgagggcgcacaggaagccatcaaggtcgccgatgccgccctctcggccgtcaacagccagcgcgctgaatacggcgcactgcagtcgcgcttcgaatcggcgatctcgaacctgtcgtcgtcgaccgagaacctgtcggcatcgcgcagccgcatcgtcgataccgacttcgcagctgaaaccgcaaagatgacccgcggccagatcctgcagcaagctggtacctcgatgctggctcaggctaactccctgccgaacggtgtcctgtcgctgctgcgtggc

SEQ ID NO: 2 - protein, 486 aa, Artificial sequence
  Synthetic construct
MASVINTNVPSLNSQRNLSTSQAQLNTSIQRLSSGMRINSAKDDAAGLAISDRMNSQIKGMTQATRNANDGVSMAQTAEGALSSSGDILQRIRELAVQSSNSSNSASDRKALQTEVTQLSSELNRIANTTEFNGQKLMDGTLGTANFQVGANAGQLISMTGANFNTSVYGNNQIAGDAAKAAATTSAAATGAFTISGYLGTSAAINTTDADTAKTIAASINNVTGDTGVTATARTDSFLKSAGGAHTMSLASDNSGTGEAVDISFTVGDGGDTADDYAAAISAFNAQTAKTGVTAEYDAVEKGLKLTNSAGENISLTNKAGSADIDMDTYAADGALMASALTLEAGDVGVANGRVTMDSEKSFSVAEGTDTGFELADSSALKSVAKIDISDFEGAQEAIKVADAALSAVNSQRAEYGALQSRFESAISNLSSSTENLSASRSRIVDTDFAAETAKMTRGQILQQAGTSMLAQANSLPNGVLSLLRG

SEQ ID NO: 3 - protein, 495 aa, Artificial sequence [sequence omitted]

SEQ ID NO: 4 - protein, 498 aa, Artificial sequence
  Synthetic construct
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG